(19) United States
(12) Patent Application Publication
(10) Pub. No.: US 2020/0306326 A1
(43) Pub. Date: Oct. 1, 2020

(54) COMPOSITIONS AND METHODS FOR THE PRODUCTION OF SHORT CHAIN FATTY ACIDS IN THE GUT

(71) Applicant: TATA CHEMICALS LIMITED, Mumbai (IN)

(72) Inventors: SUCHETA GOKHALE, PUNE (IN); ANIRBAN BHADURI, PUNE (IN)

(21) Appl. No.: 16/833,243

(22) Filed: Mar. 27, 2020

(30) Foreign Application Priority Data
Mar. 30, 2019 (IN) 201921012941

(51) Int. Cl. A61K 35/747; A61K 47/26; C12P 7/64; C12N 1/20

(52) U.S. Cl. CPC A61K 35/747; C12N 1/20; C12P 7/6409; A61K 47/26

(57) ABSTRACT

The present disclosure relates to compositions and methods for the production of short chain fatty acids in the gut of a host organism. The compositions of the disclosure are synbiotic compositions comprising the microorganism Lactobacillus rhamnosus. The present method relates to an in silico method for the rational design of synbiotic compositions.

### FIELD OF INVENTION

The present disclosure relates generally to the field of computer-implemented systems for design of therapeutic compositions. Specifically, the present disclosure relates to the rational design of synbiotic compositions.

### BACKGROUND

Synbiotic compositions refer to compositions comprising prebiotic and probiotics. Prebiotics are non-digestible selectively fermentable ingredients which help in facilitating specific changes in the microbiota present in a host organism's gut. These changes include increased growth and proliferation of the microorganisms in the gut, change in the composition of the microbiota, among others. These changes help in the health and well-being of the host organism by bringing about various metabolic changes within the host such as improved bowel movements, stimulation of mineral absorption, reducing post-prandial glucose levels, reducing risk of obesity etc. Prebiotics are primarily obtained from plant derived sources such as vegetable, root and tuber crops like onion, garlic as well as some fruit crops like dragon fruit and the prebiotic-rich grain crops like barley, chickpea, lentil, lupin and wheat. Some less commonly used sources include yeast cell wall, seaweed, and microalgae.

Probiotics are live microorganisms, which when consumed, help to promote beneficial health effects within a host organism. Probiotics are usually ingested in the form of foods such as yoghurts, fermented food products such as beverages, and dietary supplements. The most common probiotics include bacteria from the species Bifidobacterium and Lactobacillus, and yeasts such as Saccharomyces boulardii. Probiotics aid in the prevention or treatment of various diseases such as metabolic disorders like diabetes and cardiovascular diseases, digestive disorders such as irritable bowel disease and constipation, allergic disorders, immunological and inflammatory diseases among others.

All probiotics do not have the same effect as different microorganisms may have specific effects on different diseases. Further, as the microorganisms may metabolize metabolites differently, the combination of a specific prebiotic and probiotic may have very different effects within the body of the host organism.

In such a scenario, it becomes essential to test to the effect of a synbiotic composition in a host organism before recommending the composition for health conditions. The effect of these compositions are usually tested using in vitro and in vivo experiments and then the constituents of the compositions are altered through trial and error.

Such a process is time consuming and inherently error prone, as the conditions may not accurately estimate the effect of the composition within the host gut.

Therefore, there is a need to provide compositions that are ideally suited to the conditions within a host organism and methods that reduce the time taken to obtain such compositions.

### SUMMARY

In an aspect of the present invention there is provided a composition for the production of short-chain fatty acids comprising Lactobacillus rhamnosus; fructooligosaccharides; and galactooligosaccharides, wherein the w/w ratio of Lactobacillus rhamnosus: fructooligosaccharides: galatcooligosaccharides is in the range of 1:1:2-1:2:1.

In another aspect of the present invention, there is provided an in silico method for obtaining a composition for the production of short-chain fatty acids in the gut of an organism, the method comprising: receiving, by a control unit, a user input including at least one parameter associated with the composition, the at least one parameter comprising one or more prebiotics and one or more short chain fatty acids; extracting, by the control unit, from a database comprising a plurality of genomic metabolic networks of Lactobacillus rhamnosus and a plurality of sets of chemical transformation rules associated with the genomic metabolic networks, a genomic metabolic model of Lactobacillus rhamnosus corresponding to the received at least one parameter associated with the composition, wherein the genomic metabolic model is based on the plurality of genomic metabolic networks and one or more of the plurality of sets of chemical transformation rules; and obtaining, by the control unit, the composition having a ratio of one or more of the prebiotics to Lactobacillus rhamnosus, based on the extracted genomic metabolic model of Lactobacillus rhamnosus.

In a further aspect of the present invention, there is provided a system for obtaining a composition for the production of short-chain fatty acids comprising: a display unit; a database comprising a genomic metabolic model of Lactobacillus rhamnosus, a plurality of genomic metabolic networks of Lactobacillus rhamnosus and a plurality of chemical transformation rules associated with the metabolic networks; a control unit operatively coupled to the display unit and the database, the processor being configured to: receive a user input including at least one parameter associated with the composition, the at least one parameter comprising one or more prebiotics, and one or more short chain fatty acids; extract the genomic metabolic model of Lactobacillus rhamnosus from the database; and obtaining a composition comprising a ratio of one or more of the prebiotics to Lactobacillus rhamnosus based on the user input and the genomic metabolic model of Lactobacillus rhamnosus.

### BRIEF DESCRIPTION OF DRAWINGS

FIG. 1 illustrates a block diagram of an exemplary data processing system 100 for processing data relating to obtaining a composition to produce short-chain fatty acids.

FIG. 2 illustrates an exemplary method 200 for obtaining a composition to produce short-chain fatty acids.

FIG. 3 illustrates the process 300 of generating the genomic metabolic model of Lactobacillus rhamnosus

FIG. 4 illustrates the method 400 of applying one or more sets of chemical transformation rules to the consensus metabolic model to obtain the genomic metabolic model of Lactobacillus rhamnosus.

FIG. 5 illustrates the steps for the method 500 for generating a composition to produce short-chain fatty acids.

FIG. 6A represents all the metabolic pathways present within a genomic metabolic network of Lactobacillus rhamnosus

FIG. 6B represents the metabolic reactions occurring within Lactobacillus rhamnosus for the production of acetate and lactate from FOS and GOS

FIG. 7 demonstrates compositions comprising the probiotic Lactobacillus rhamnosus and a combination of the prebiotics FOS and GOS

FIGS. 8A-8C demonstrate the optimal composition ratios of Lactobacillus rhamnosus: FOS: GOS for the production of acetate and lactate

FIG. 9 demonstrates the effect of using different species of Lactobacillus with the prebiotics FOS and GOS in a ratio of 1:1 for production of acetate and lactate

FIGS. 10A-10C demonstrates the fluxes of acetate and lactate for FOS and GOS correlated with the growth of Lactobacillus rhamnosus

FIG. 11 shows the correlation of the results obtained from the method of the present disclosure with in vitro experiments

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are explanatory of the invention and are not intended to be restrictive thereof.

The terms “a,” “an,”, and “the” are used to refer to “one or more” (i.e. to at least one) of the grammatical object of the article.

Reference throughout this specification to “an aspect”, “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion and are not intended to be construed as “consists of only”, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Likewise, the terms “having” and “including” and their grammatical variants are intended to be non-limiting, such that recitations of said items in a list is not to the exclusion of other items that can be substituted or added to the listed items.

The term flux, or metabolic flux is the rate of turnover of molecules through a metabolic pathway. Flux is regulated by the enzymes involved in a pathway. The regulation of flux is vital for all metabolic pathways to regulate the pathway's activity under different conditions.

Prebiotics are non-digestible selectively fermentable ingredients which help in facilitating specific changes in the microbiota present in a host organism's gut. Examples of prebiotics include Galactooligosaccharides (GOS), Fructooligosaccharides (FOS), inulin, resistant starch, dextrin, Xylooligosaccharides (XOS), mannan-oligosaccharide (MOS) among others.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

In the present disclosure there is provided a composition for the production of short-chain fatty acids comprising Lactobacillus rhamnosus: fructooligosaccharides; and galactooligosaccharides, wherein the weight/weight ratio of Lactobacillus rhamnosus: fructooligosaccharides: galatcooligosaccharides is in the range of 1:1:2-1:2:1.

In an exemplary embodiment, there is provided a composition for the production of short-chain fatty acids comprising Lactobacillus rhamnosus; fructooligosaccharides; and galactooligosaccharides, wherein the weight/weight ratio of Lactobacillus rhamnosus: fructooligosaccharides: galatcooligosaccharides is 1:1:2.

In a further embodiment, the composition is provided for the production of the short chain fatty acids selected from the group consisting of acetate, lactate, propionate, butyrate and combinations thereof. In a preferred embodiment, the short chain fatty acids are acetate, lactate, and combinations thereof.

In another embodiment of the present disclosure, the composition comprises L. rhamnosus and a combination of fructooligosaccharides and galactooligosaccharides in a ratio of 1:2 for the production of acetate. Further, the composition comprising L. rhamnosus and a combination of fructooligosaccharides and galactooligosaccharides is in the preferred ratio of 1:4 for the production of lactate.

In an embodiment, the composition comprises a w/w ratio of 1:1:1 of Lactobacillus rhamnosus: fructooligosaccharides: galatcooligosaccharides for the production of acetate. Further, the composition comprises a w/w ratio of 1:2:2 of Lactobacillus rhamnosus: fructooligosaccharides: galatcooligosaccharides for the production of lactate.

In a further embodiment, the composition of the present disclosure may be in the form of a liquid formulation, a solid formulation, or a semi-solid formulation. It may be provided in the form of a tablet, or a capsule, a dietary supplement, or an energy drink.

In an aspect of the present disclosure, the compositions described herein are obtained through methods and systems comprising in silico processes. Specifically, the present disclosure relates to an in-silico method for obtaining a composition to produce short-chain fatty acids using Lactobacillus rhamnosus, the method comprising:

receiving, by a processor, a user input including at least one parameter associated with the composition, the at least one parameter comprising one or more prebiotics and one or more short chain fatty acids;

extracting, by the processor from a database comprising a plurality of genomic metabolic networks of Lactobacillus rhamnosus and a plurality of sets of chemical transformation rules associated with the genomic metabolic networks, a genomic metabolic model of Lactobacillus rhamnosus corresponding to the received at least one parameter associated with the composition, wherein the genomic metabolic model is based on the plurality of genomic metabolic networks and one or more of the plurality of sets of chemical transformation rules; and

obtaining, by the processor, the composition having a ratio of one or more of the prebiotics to Lactobacillus rhamnosus, based on the extracted genomic metabolic model of Lactobacillus rhamnosus.

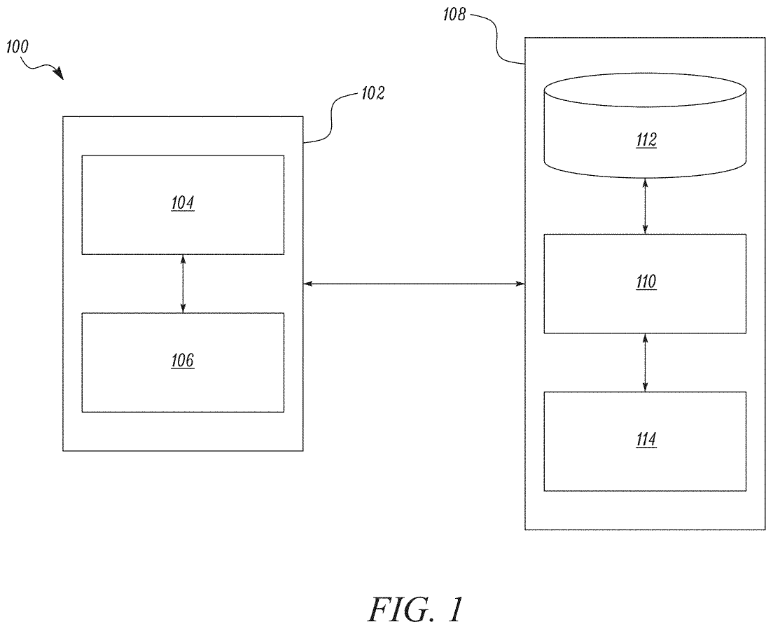

FIG. 1 illustrates a block diagram of an exemplary data processing system 100 for processing data relating to obtaining one or more nutraceutical compositions. The data processing system 100 depicted in FIG. 1 may be implemented in any suitable computing environment, such as, a desktop or laptop computer, a computer server, or a mobile computing device, such as a mobile phone, a Personal Digital Assistant (PDA), or a smart phone. In addition, the data processing system 100 may be combined into fewer systems than shown, or divided into more systems than shown. The communications links depicted in FIG. 1 may be through wired or wireless connections and may be part of a secured network, such as a local area network (LAN) and/or a combination of networks, such as LANs, WANs, MANs and/or the Internet.

According to an embodiment of the present disclosure, the data processing system 100 includes an Input/ Output unit 102, hereinafter referred to as I/O unit 102. The I/O unit includes an output unit 104 and an input unit 106. The output unit 104 may include a display unit having a screen such as one or more of a computer screen, a mobile screen, or a television screen. The output unit 104 serves as a means for a user to visualize data that has may be entered by the user using the input unit 106, and to optionally visualize data that may be generated by a control unit 108.

The input unit 106 serves as means for the user to input values and provide instructions to the control unit 108 for processing data associated with the inputted values. The input unit 106 also serves as means for the user to manipulate, study, and the screen data, received from the control unit 108. In one embodiment, the input unit 106 may include a keyboard and/or a mouse, a joystick. Alternatively, or in combination with the keyboard and/or the mouse, the input unit 106 may also be a hands free voice-controlled device. In another embodiment, the input unit 106 and the output unit 104 may be part of the same device, and may take the form of a device, such as a tablet, a mobile, or touchscreen computer. The I/O unit may be operatively linked to the control unit 108.

The control unit 108 includes a processing unit 110. The processing unit 110 may include microprocessors, microcomputers, microcontrollers, digital signal processors, central processing units, state machines, logic circuitries, and/or other devices. The control unit 108 also includes a communication unit 114. The communication unit 114 may include a modem, an Ethernet card, or other similar devices, which enable the control unit 108 to connect to databases and networks. The control unit 108 facilitates input from a user through the Input/Output unit 102.

Among other capabilities, the processing unit 110 may fetch and execute programmable or computer-readable instructions. One or more programmable or computer-readable instructions may include various commands that instruct the control unit 108 to perform specific tasks, such as steps that constitute the method of the disclosure. The processing unit 110 described may also be implemented using only software programming or using only hardware or by a varying combination of the two. The computer-readable instructions may be written in programming languages including, but not limited to, ‘C’, ‘C++’, ‘Visual C++’ and ‘Visual Basic’. Further, the software may be in the form of a collection of separate programs, a program module containing a larger program or a portion of a program module, as discussed in the ongoing description. The software may also include modular programming in the form of object-oriented programming. The processing of input data by the processing machine may be in response to user commands, the results of previous processing, or from a request made by another processing machine. Aspects of the disclosure can be implemented in various operating systems and platforms including, but not limited to, ‘Unix’, DOS', ‘Android’, ‘Symbian’, and ‘Linux’.

The computer-readable instructions can be stored and transmitted via a computer-readable medium. The disclosure can also be embodied in a computer program product including a computer-readable medium, or with any product capable of implementing the above methods and systems, or the numerous possible variations thereof.

Among other capabilities, the processing unit 110 may fetch and execute computer-readable instructions stored in a database 112 coupled to the processing unit 110. The database 112 can be internal or external to the control unit 108 and maybe accessed through cloud computing. The database 112 may include any non-transitory computer-readable storage medium including, for example, volatile memory (e.g., RAM), or non-volatile memory comprising a storage device such as a hard disk drive, or a removable storage drive, such as, a floppy-disk drive, optical-disk drive, and the like. The storage device may also be a means for loading computer programs or other instructions into the computer system.

The database 112 includes information regarding a plurality of genomic metabolic networks of Lactobacillus rhamnosus, a plurality of prebiotics, and a plurality of markers associated with a plurality of health conditions and one or more metabolic pathways of Lactobacillus rhamnosus.

Genomic metabolic networks of Lactobacillus rhamnosus refer to mathematical reconstructions of all the known metabolic reactions occurring within Lactobacillus rhamnosus. This information of the complete set of metabolic reactions is correlated to the sequences of those genes and proteins known to have functions in the metabolic reactions of Lactobacillus rhamnosus. The reconstructions, therefore, collect all the information regarding the metabolic processes within the microorganism, and represent it in the form of a mathematical network. The metabolic networks are stored in the database in the form of a metabolite-reaction matrix representing all the metabolites that are processed or exchanged by the reactions occurring within Lactobacillus rhamnosus. In an example implementation, every row of the matrix represents one unique metabolite and every column represents one reaction. The entries in each column are the stochiometric coefficients of the metabolites participating in the reaction. In this manner, the metabolite-reaction matrix specifies a plurality of reaction constraints for metabolic pathways in the genomic metabolic networks of Lactobacillus rhamnosus.

One or more of such metabolite-reaction matrices may be obtained from publicly available databases and literature. Examples of such sources include BiGG Models, BioCyc, MetaNetX.org, Biomodels database, VMH database, Machado, Daniel, et al., Nucleic acids research 2018, 46.15, 7542-7553. The user may also periodically update the metabolite-reaction matrices with further data as and when new and more accurate data is obtained. These data may comprise experimental data obtained through privileged and/or personal communications, published experimental data, or updates to the data sets available from publicly available databases. Updates on the genomic metabolic networks may be done manually by the user or through an automated program that periodically accesses selected databases to download recent updates to genomic networks or their associated metabolic reaction pathways.

In a further embodiment, the database 112 also comprises a genomic metabolic model of Lactobacillus rhamnosus. This genomic metabolic model is built by the control unit 108 by integrating a plurality of the genomic metabolic networks of Lactobacillus rhamnosus. Once the control unit 108 builds the genomic metabolic model, the control unit 108 stores the model in the database 112, for future use.

In an embodiment of the present disclosure, the database 112 comprises information regarding prebiotics. The prebiotics described herein are associated with the metabolic pathways Lactobacillus rhamnosus, such that these prebiotics are readily metabolised by the enzymes and enzymatic reactions occurring within Lactobacillus rhamnosus. The maximum and minimum flux values of each prebiotic is included in the database and correlated with the metabolic pathways in which each of the plurality of prebiotics may be metabolized. In this manner, the reaction constraints for each of the metabolic pathways and the associated prebiotic(s), are stored within the database, thus providing estimates for the metabolism of such molecules within the gut of a host organism. Data pertaining to the flux values and additional data such as recommended dietary allowances (RDA) can be obtained through experimental values obtained through experimentation and/or published literature and databases such as FooDB, United States Envrionmental Protection Agency—Chemistry Dashboard, Chemical Entities of Biological Interest (ChEBI), Kyoto Encyclopedia of Genes and Genomes (KEGG), METLIN, Pubchem, KNApSAcK, BRENDA, KO (KEGG Orthology) database. In an embodiment, the prebiotics included in the database maybe selected from the group consisting of Galactooligosaccharides (GOS), Fructooligosaccharides (FOS), inulin, resistant starch, dextrin, Xylooligosaccharides (XOS), mannan-oligosaccharide (MOS), and combinations thereof.

In another embodiment of the present disclosure, the database 112 also comprises of markers associated with one or more metabolic pathways of Lactobacillus rhamnosus. Markers included in the database include molecules and compounds comprising short chain fatty acids such as, butyrates, propionate, acetate, lactate, lipids, carbohydrates, bile salts, siderophores, insulin, and combinations thereof. These molecules are well known indicators of specific health conditions. Examples of such health conditions include metabolic disorders comprising obesity, Cardiovascular Disease, and Type I and Type II diabetes, immunological disorders such as inflammatory bowel diseases, Crohn's disease, and irritable bowel syndrome, food allergies, asthma, acute infections, neurological disorders comprising depression, and anxiety, among others. Lactobacillus rhamnosus populations residing within in the gut of an organism are known to influence the levels of these metabolic markers. They are capable of metabolising or releasing these molecules and therefore changing the levels of these markers correspondingly within a host organism's body. Additionally, factors such as the presence of a disease, food intake habits, consumption of antibiotics, and other such factors are known to affect the microbial physiological activity within the gut, thereby exacerbating or resulting in various health conditions such as those listed herein. The same markers can be used to assess whether chosen composition parameters are likely in aiding in alleviating certain health conditions. Additionally, these markers can also be used to assess whether chosen composition parameters are able to promote the long term health and wellness of a host organism.

According to an implementation of the present disclosure, the database 112 comprises a plurality of sets of chemical transformation rules. The chemical transformation rules also include reaction steps and reaction constraints of molecules, and therefore the chemical transformation rules can be extrapolated to similar molecular species for predicting similar reaction pathways. The chemical transformation rules, as described herein, comprise reactions associated with the metabolic pathways of Lactobacillus rhamnosus. More specifically, the chemical transformation rules are derived from enzymes within the microorganism. Chemical transformation rules include biochemical reaction transformation rules and general chemical rules that are well understood and familiar to a person skilled in the art. In general, these chemical transformation rules relate to the mechanism of action, and the criteria relevant for the progress of different biochemical reactions occurring in the metabolic pathways of Lactobacillus rhamnosus. These mechanisms of action can then be extrapolated to similar metabolites involved in these biochemical reactions, thereby allowing for the prediction of the progression and outcome of the biochemical reaction. The chemical transformation rules can therefore be used for two-way prediction of reactions in silico, i.e. both forward and retrosynthetic reactions can be predicted accurately. The chemical transformation rules are added to the database and are based on experimental data and publicly available literature. An exemplary approach to biochemical pathway prediction using chemical transformation rules is described in Sivakumar et al., 2016; Bioinformatics, 32, 3522-3524.

In an embodiment of the present disclosure, the database 112 maybe a single database or may comprise multiple databases that may be located in different locations. The multiple databases may store all of the components of the information described above or may comprise parts of the information described above. For instance, a first database may store the information related to Lactobacillus rhamnosus, a second database may store information related to the prebiotics, and a third database may store information related to the metabolic markers.

In some embodiments, the databases may be implemented using a relational database, such as Sybase, Oracle, CodeBase and Microsoft® SQL Server as well as other types of databases such as, for example, a flat file database, an entity-relationship database, and object-oriented database, a record-based database, or the like.

Those skilled in the art will appreciate that any of the aforementioned steps and/or system modules may be suitably replaced, reordered, or removed, and additional steps and/or system modules may be inserted, depending on the needs of a particular application, but without departing from the scope and spirit of the disclosure. In addition, the systems of the aforementioned embodiments may be implemented using a wide variety of processes and system modules not discussed herein, and is thus not limited to any particular computer hardware, software, middleware, firmware, microcode, or the like.

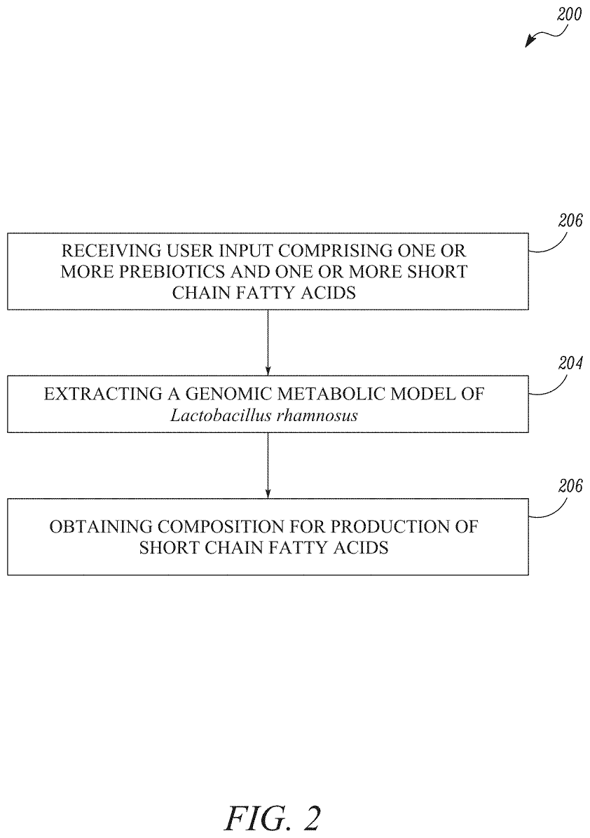

FIG. 2 illustrates an exemplary method 200 performed by the data processing system 100 of the present disclosure. At step 202 a user input comprising the prebiotics, and short chain fatty acids, is received by the control unit 108.

At step 204 the genomic metabolic model of Lactobacillus rhamnosus is extracted from the database 108 and at step 206 a composition that is based on the extracted genomic metabolic model is generated by the control unit 108. Further, in an embodiment, a plurality of compositions may also be generated allowing the user to screen and select the composition with the most optimal effect on the production of acetate and lactate.

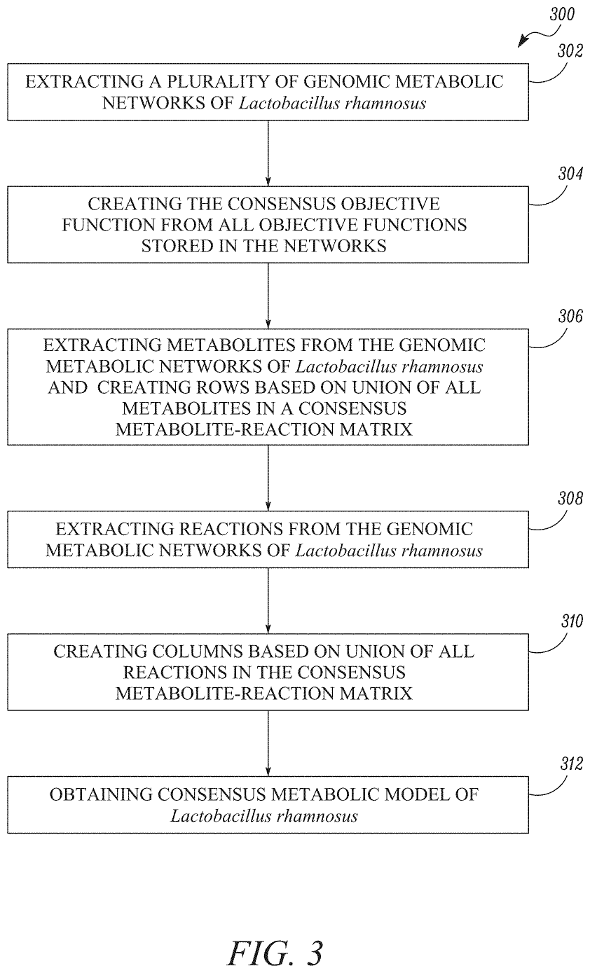

FIG. 3 illustrates the process 300 of generating the genomic metabolic model of Lactobacillus rhamnosus. At step 302 a plurality of genomic metabolic networks of Lactobacillus rhamnosus are obtained and used at step 304 to create a consensus objective function from all the objective functions stored in the models.

The objective function in the genomic metabolic networks is a set of one or more optimization functions which aims to either maximize or minimize the flux through a set of metabolites that is either processed or internalized by the microorganism and the markers of the one or more health conditions. The maximisation function is represented through either linear or polynomial inequalities for the one or more metabolites of the microorganisms.

At step 306 the metabolites from the genomic metabolic networks are extracted and added to a consensus metabolite-reaction matrix, the matrix representing the combination of the information from all the metabolic networks. Rows are created in the consensus metabolite-reaction matrix based on the union of all the metabolites from all the genomic metabolic networks.

At step 308 the metabolic reactions are extracted from the genomic metabolic networks and at step 310 columns are created in the consensus metabolite-reaction matrix based on the union of the all the metabolic reactions that are extracted.

At step 312 a consensus metabolic model of Lactobacillus rhamnosus is obtained from the steps described above.

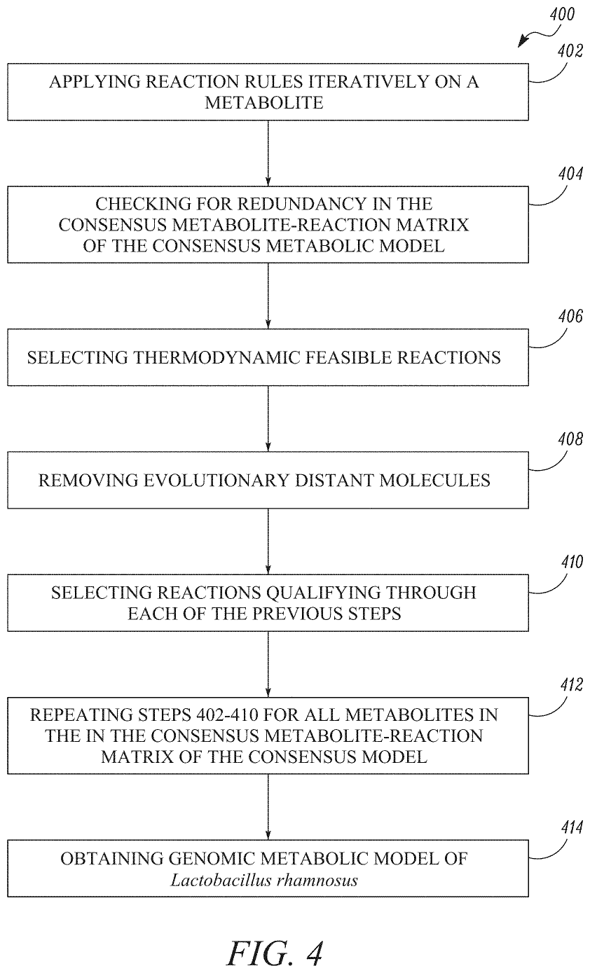

FIG. 4 illustrates the method 400 of applying one or more sets of chemical transformation rules to the consensus metabolic model to obtain the genomic metabolic model of Lactobacillus rhamnosus.

Once the consensus metabolic model is obtained, at step 402 one or more sets of chemical transformation rules are applied iteratively on a specific metabolite. At step 404 the consensus metabolite-reaction matrix of the model is checked for redundancy of the metabolite and the reactions pertaining to it. At step 406 the thermodynamically feasible reactions pertaining to the metabolite are selected in the metabolite-reaction matrix.

At step 408 those molecules that are evolutionary distant molecules are removed from the reactions. At step 410 only those reactions qualifying through each of the previous steps are included and at step 412 the previous steps 402-410 are repeated for all the metabolites in the consensus metabolite-reaction matrix of the consensus model. Finally, once the processing steps are performed for the metabolic reactions in the model, at step 414 the genomic metabolic model of Lactobacillus rhamnosus is obtained.

This genomic metabolic model is considerably optimized from all the previous networks related to Lactobacillus rhamnosus as any gaps in the information related to the metabolic networks are filled using first, the steps involved in forming the consensus metabolic model, and then through the steps of the application of the chemical transformation rules which are able to predict reactions which may be incomplete or absent in the models.

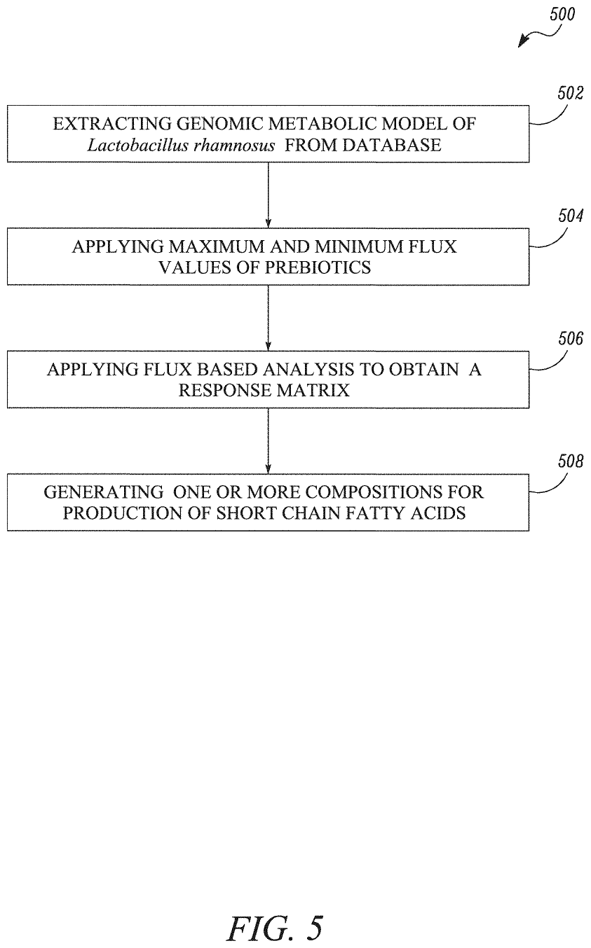

FIG. 5 illustrates the steps for the method 500 for generating the composition described herein. Referring to step 502, the genomic metabolic model generated through the methods 300 and 400 is extracted. As in the case of the genomic metabolic networks, the genomic metabolic model is stored in the form of a metabolite-reaction matrix that comprises all the reaction constraints associated with metabolic reactions represented in the metabolite-reaction matrix. When extracted, the metabolite-reaction matrix of the network is obtained along with the associated set of chemical transformation rules.

At step 504 the maximum and minimum flux values of the prebiotics are applied to one or more rows of the metabolite-reaction matrix of the metabolic model.

At step 506 a constraints-based application is applied to the genomic metabolic model of Lactobacillus rhamnosus and the prebiotics to obtain a response matrix that describes the flux distribution that maximises or minimises the levels of the short chain fatty acids. The response matrix may be stored in the database 112.

In one aspect, a constraints-based application is the preferred methodology to obtain the flux distribution results. The most commonly used constraints-based methodologies applicable to the method described herein include, but are not limited to flux balance analysis (FBA), regulatory flux balance analysis (rFBA), flux variability analysis (FVA), minimization of metabolic adjustment (MoMA), and regulatory on-off minimization (ROOM). Most preferably, flux based analysis is used.

At step 508 the control unit 108 screens the response matrix for the optimal dosage amounts of the composition and reports the optimal compositions or, alternatively, the user may screen the response matrix and select the optimal composition through the I/O unit 102.

The invention will now be explained in greater detail with the help of the following non-limiting examples. It is to be appreciated that the examples are intended to be exemplary only and that numerous changes, modifications, and alterations can be employed without departing from the scope of the presently disclosed subject matter.

### EXAMPLES

Developing the Genomic Metabolic Network of Lactobacillus rhamnosus

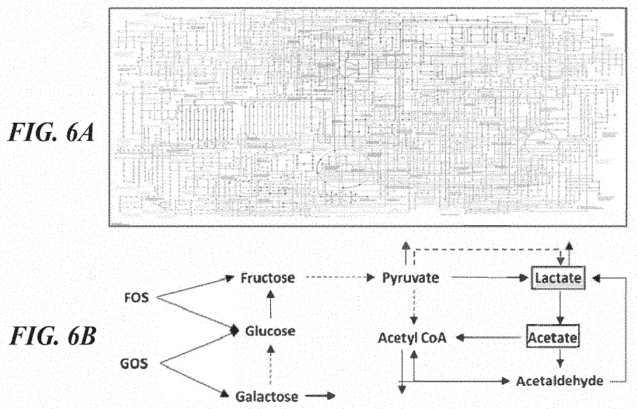

A plurality of genomic metabolic networks of Lactobacillus rhamnosus, such as the one shown in FIG. 6a were extracted from a database by a control unit as described in FIG. 1. FIG. 6a illustrates a genomic metabolic network that comprises all of the known metabolic reactions occurring within Lactobacillus rhamnosus. Once such reactions were obtained from a plurality of genomic metabolic networks, a consensus objective function from all the objective functions stored in the models was created.

The metabolites from the genomic metabolic networks were extracted from the database and added to a consensus metabolite-reaction matrix. Rows were created in the consensus metabolite-reaction matrix based on the union of all the metabolites from all the genomic metabolic networks. Next, the metabolic reactions were extracted from the genomic metabolic networks and columns were created in the consensus metabolite-reaction matrix based on the union of the all the metabolic reactions that were extracted. Following this, a consensus metabolic model of Lactobacillus rhamnosus was obtained.

This consensus model was then optimized by using one or more chemical transformation rules that help to fill any data, regarding these metabolic reactions. Once the consensus metabolic model is obtained, one or more sets of chemical transformation rules were applied iteratively on a specific metabolite. The consensus metabolite-reaction matrix of the model was checked for redundancy of the metabolite and the reactions pertaining to it. The thermodynamically feasible reactions pertaining to the metabolite were selected in the metabolite-reaction matrix, based on group contribution method. Next, evolutionary distant molecules having a Tanimoto coefficient less than 0.7, were removed from the reactions. Only those reactions qualifying through each of the previous steps were included and the previous steps were repeated for all the metabolites in the consensus metabolite-reaction matrix of the consensus model. Finally, once the processing steps were performed for the metabolic reactions in the model, the genomic metabolic model of Lactobacillus rhamnosus was obtained.

### Generation of Composition for Production of Acetate and Lactate

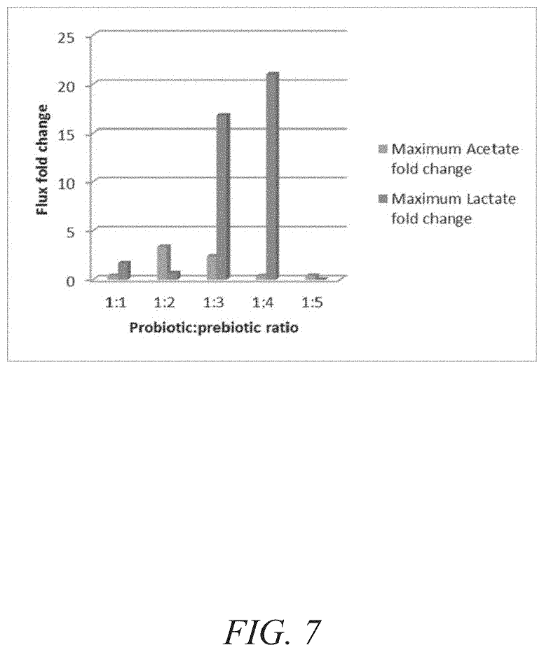

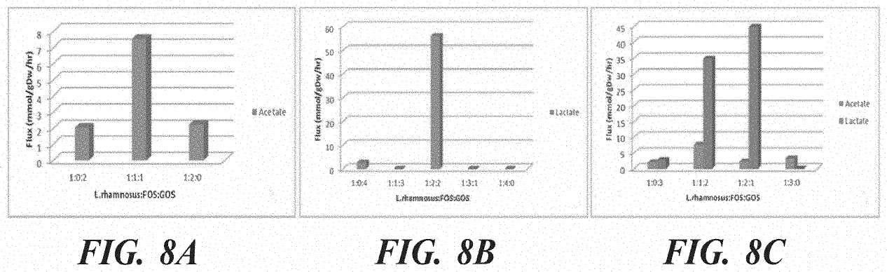

A user input specifying the prebiotics FOS and GOS for the maximum production of the short chain fatty acids acetate and lactate was given and the model was extracted from the database. The metabolic reactions occurring within Lactobacillus rhamnosus for the production of acetate and lactate from FOS and GOS is described in FIG. 6b. Flux based analysis was applied on the genomic metabolic model of Lactobacillus rhamnosus to optimize the production of acetate and lactate in the presence of FOS and GOS. Flux based analysis identifies the optimum composition parameters and these are reported to the user. FIG. 7 shows one such result where the different ratios of Lactobacillus rhamnosus with the combination of FOS and GOS affect the flux of acetate and lactate. FIG. 8 shows the optimal composition ratios of Lactobacillus rhamnosus: FOS: GOS for the production of acetate and lactate derived from the above described method. As is seen from FIG. 8c, that a w/w ratio of 1:1:2 is ideal for the production of both acetate and lactate.

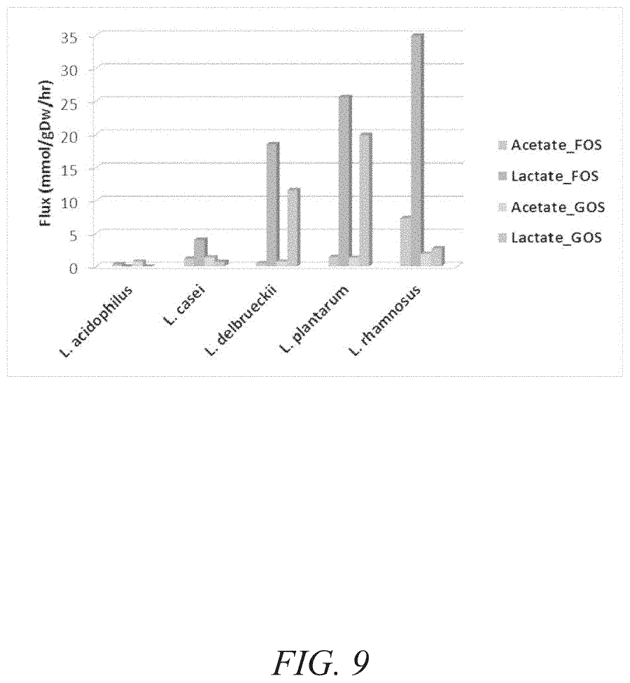

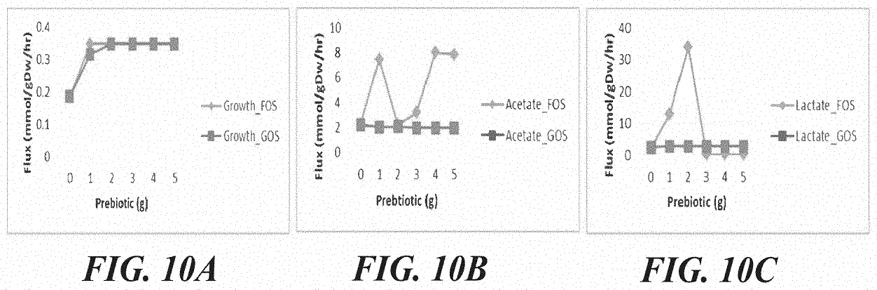

The present composition is unique in that only the specific combination of Lactobacillus rhamnosus, FOS, and GOS is able to produce the optimal level of acetate and lactate. As shown in FIG. 9, replacing Lactobacillus rhamnosus with any other species of Lactobacillus does not provide the same results. Further, as demonstrated in FIG. 10, a poor correlation is observed between the growth of Lactobacillus rhamnosus and acetate or lactate production. Therefore, the present composition does not function through the increased proliferation of the Lactobacillus rhamnosus in the gut of the host microorganism.

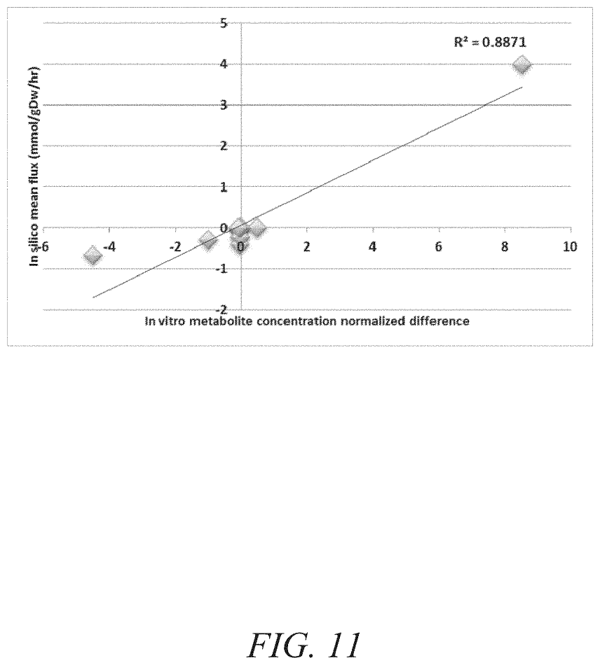

The present composition is also obtained through a unique in silico method that is able accurately mimic in vivo and in vitro experimental conditions. This is achieved through the use of chemical transformation rules to optimize the genome metabolic model of Lactobacillus rhamnosus which enables better representation of all the metabolic reactions in the genome of the bacterium and therefore facilitates more accurate flux based analysis results. The accuracy of the present method is demonstrated in in FIG. 11 which shows very high correlation between the results obtained from in vitro experiments and the results obtained from the method described herein.

While aspects of the present disclosure have been particularly shown, and described with reference to the embodiments above, it will be understood by those skilled in the art that various additional embodiments may be contemplated by the modification of the disclosed machines, systems, and methods without departing from the spirit and scope of what is disclosed. Such embodiments should be understood to fall within the scope of the present disclosure as determined based upon the claims and any equivalents thereof.

We claim:

1. A composition for the production of short-chain fatty acids comprising Lactobacillus rhamnosus; fructooligosaccharides; and galactooligosaccharides,
wherein the w/w ratio of Lactobacillus rhamnosus: fructooligosaccharides: galatcooligosaccharides is in the range of 1:1:2-1:2:1.

2. The composition as claimed in claim 1, wherein the w/w ratio of Lactobacillus rhamnosus: fructooligosaccharides: galatcooligosaccharides is 1:1:2.

3. The composition as claimed in claim 1, wherein the short chain fatty acid is acetate, and acetate is produced at a w/w ratio of 1:1:1 of Lactobacillus rhamnosus: fructooligosaccharides: galatcooligosaccharides.

4. The composition as claimed in claim 1, wherein the short chain fatty acid is lactate, and lactate is produced at a w/w ratio of 1:2:2 of Lactobacillus rhamnosus: fructooligosaccharides: galatcooligosaccharides.

5. An in silico method for obtaining a composition for the production of short-chain fatty acids in the gut of an organism, the method comprising:
receiving, by a control unit, a user input including at least one parameter associated with the composition, the at least one parameter comprising one or more prebiotics and one or more short chain fatty acids;
extracting, by the control unit, from a database comprising a plurality of genomic metabolic networks of Lactobacillus rhamnosus and a plurality of sets of chemical transformation rules associated with the genomic metabolic networks, a genomic metabolic model of Lactobacillus rhamnosus corresponding to the received at least one parameter associated with the composition, wherein the genomic metabolic model is based on the plurality of genomic metabolic networks and one or more of the plurality of sets of chemical transformation rules; and
obtaining, by the control unit, the composition having a ratio of one or more of the prebiotics to Lactobacillus rhamnosus, based on the extracted genomic metabolic model of Lactobacillus rhamnosus.

6. The method as claimed in claim 5, wherein the database further comprises:
a plurality of reaction constraints for metabolic pathways of Lactobacillus rhamnosus; and
a plurality of markers associated with the metabolic pathways of Lactobacillus rhamnosus.

7. The method as claimed in claim 5, wherein extracting, by the control unit, comprises:
extracting, by the control unit from the database, the plurality of genomic metabolic networks of Lactobacillus rhamnosus;
combining, by the control unit, the plurality of genomic metabolic networks into a consensus genomic metabolic network of Lactobacillus rhamnosus; and
applying, by the control unit, one or more of the sets of chemical transformation rules to the consensus genomic metabolic network to obtain the genomic metabolic model of Lactobacillus rhamnosus.

8. The method as claimed in claim 5, wherein obtaining, by the control unit, comprises:
extracting, by the control unit from the database, the genomic metabolic model of Lactobacillus rhamnosus;
extracting, by the control unit from the database, the plurality of reaction constraints for metabolic pathways of Lactobacillus rhamnosus;
integrating, by the control unit, one or more of the plurality of reaction constraints for the metabolic pathways to the genomic metabolic model of Lactobacillus rhamnosus; and
applying, by the control unit, the received one or more parameters associated with the composition based on the user input, to determine the composition to produce the short-chain fatty acids.

9. The method as claimed in claim 5, wherein the prebiotics are selected from a group consisting of galactooligosaccharides, fructooligosaccahrides, inulin, resistant starch, dextrin, xylooligosaccharides (XOS), mannan-oligosaccharide (MOS), and combinations thereof.

10. The method as claimed in claim 5, wherein the prebiotics comprise galactooligosaccharides and fructooligosacharides.

11. The method as claimed in claim 5, wherein the short-chain fatty acids include acetate, lactate, propionate, and combinations thereof.

12. The method as claimed in claim 5, wherein the composition comprises Lactobacillus rhamnosus: fructooligosaccharides: galatcooligosaccharides in a w/w ratio in the range of 1:1:2-1:2:1.

13. The method as claimed in claim 5, wherein the composition comprises Lactobacillus rhamnosus: fructooligosaccharides: galatcooligosaccharides in a w/w ratio of 1:1:2.

14. A system for obtaining a composition for the production of short-chain fatty acids comprising:
a display unit;
a database comprising a genomic metabolic model of Lactobacillus rhamnosus, a plurality of genomic metabolic networks of Lactobacillus rhamnosus and a plurality of chemical transformation rules associated with the metabolic networks;
a control unit operatively coupled to the display unit and the database, the processor being configured to:
receive a user input including at least one parameter associated with the composition, the at least one parameter comprising one or more prebiotics, and one or more short chain fatty acids;
extract the genomic metabolic model of Lactobacillus rhamnosus from the database; and
obtaining a composition comprising a ratio of one or more of the prebiotics to Lactobacillus rhamnosus based on the user input and the genomic metabolic model of Lactobacillus rhamnosus.
